(19) United States
(12) Patent Application Publication
(10) Pub. No.: US 2012/0190842 A1
(43) Pub. Date: Jul. 26, 2012

(54) PROCESS FOR THE PREPARATION OF ANTIBIOTIC COMPOUNDS

(75) Inventors: Wei-Hong Tseng, Miaoli County (TW); Wen-Hsin Chang, Miaoli County (TW); Shiuan-Ting Chuang, Miaoli County (TW)

(73) Assignee: SAVIOR LIFETEC CORPORATION, Miaoli County (TW)

(21) Appl. No.: 13/012,171

(22) Filed: Jan. 24, 2011

(51) Int. Cl. C07D 487/04; F26B 5/06

(52) U.S. Cl. 540/350; 34/284

(57) ABSTRACT

The present invention relates to a process for the preparation of carbapenem antibiotic compounds, which is useful for intravenous and intramuscular administration.

### BACKGROUND OF THE INVENTION

1. Field of the Invention

The present invention relates to a simplified process for preparing a product of carbapenem antibiotic compounds.

2. The Prior Arts

Carbapenem is a class of β-lactam antibiotics having a broader spectrum of antibacterial activity than other β-lactam antibiotics. The formula (I) has an unusual structure of carbapenem, which renders itself strongly resistant to typical bacterial beta-lactamse enzymes. In other words, carbapenem is able to be used as the last resort for many serious bacterial infections including gram positive and negative, aerobic and anaerobic bacteria. However, unstable propriety of carbapenem brings about a problem in commercially manufacturing. As the environmental temperature goes up, accelerating dimerization and hydrolysis deteriorate the quality of carbapenem. Take ertapenem for example, it is unstable above −20° C. and must be stored at a low temperature. Therefore, several researches disclose how to achieve a stable form of carbapenem antibiotics in its formulation and manufacturing process. In particular, several processes for conversion of salt-containing carbapenem to a formulation of the compound of formula (II) have been reported.

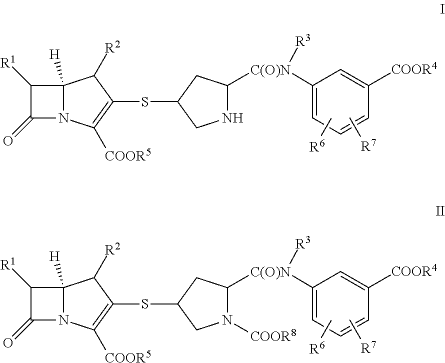

WO2001/32172 A1 describes a process with detail steps for converting ertapenem monosodium into a stable formulation. The whole process contains more than 10 steps. Even though WO2002/34750 A1 describes a similar formulation process for carbapenem antibiotics comprising the following steps of: (1) charging a solution of carbon dioxide source having a pH range of about 6.0˜42.0; (2) adding an effective amount of a mole ratio of a base and active ingredient into the reaction vessel containing the solution of carbon dioxide source to maintain pH at about 6.0 to 9.0 and a temperature of about −3° C. to about 15° C.; (3) lyophilizing the solution of step (2) to yield the final formulation product of a compound of formula (I) with less than about 10% of moisture content. The actual manufacturing process of the later patent still follows more than 10 steps, including charging water for injection 3 times, weighting, carefully maintaining a pH range by alternately adding carbapenem and base.

With our continued research for developing a simplified process for converting carbapenem or its pharmaceutically acceptable salt, hydrate, or solvate to a final formulation product of carbapenem antibiotic with acceptable levels of degradates, solid state stability and solution stability for dosing. The present invention is not only commercially viable, but also involves simplifying the manufacturing process avoiding multiple water charging and titration.

### SUMMARY OF THE INVENTION

The objective of the present invention is to provide a simple and commercially viable process for manufacturing a stable product of carbapenem antibiotic compound of the formula (I).

Accordingly, the present invention provides a improved process for manufacturing a solution of a compound of formula (I):

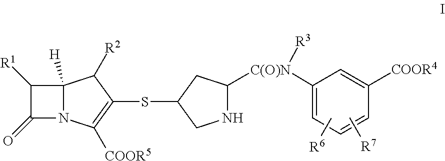

or its pharmaceutically acceptable salt, hydrate or solvate;
wherein, R¹ is 1-hydroxyethyl, 1-fluoroethyl, or hydroxymethyl;
R² and R³ are independently hydrogen, or C₁-C₆ alkyl;
R⁴ and R⁵ are independently hydrogen, C₁-C₆ alkyl, or alkali-metal or alkali earth-metal wherein the alkali-metal or alkali earth-metal is sodium, potassium, lithium, cesium, rubidium, barium, calcium or magnesium;
R⁶ and R⁷ are independently hydrogen, halo, cyano, nitro, hydroxy, carboxy, amino, C₁-C₆ alkylamino, di C₁-C₆ alkylamino, C₁-C₆ alkoxy, C₁-C₆ alkoxycarbonyl, aminosulphonyl, C₁-C₆ alkylaminosulphonyl, di-C₁-C₆ alkylaminosulphonyl, carbamoyl, C₁-C₆ alkylcarbamoyl, trifluoromethyl, sulphonic acid, sulphonic acid, C₁-C₆ alkanoylamino, C₁-C₆ alkanoyl(N—(C₁-C₆)-alkyl)amino, C₁-C₆ alkanesulphonamido, C₁-C₆ alkyl-S(O)ₙ wherein n is 0-2;
comprising the steps of:
(a) dissolving a carbonate source and a base in a diluent to form a first solution at a temperature from 0° C. to 25° C., wherein a mole ratio of the carbonate source to the compound of formula I is 0.5 to 1.5, and a mole ratio of the base to the compound of formula I is 0.1 to 1.0; and
(b) mixing the compound of formula I with the first solution at a temperature from −5° C. to 25° C. to form the solution.

The process optimizes a process by the following modification: reducing over 10-step manufacturing, simplifying multiple titrations and loosing up the in-process restrictions. The pH naturally falls at the appropriate range from about 6.5 to about 8.5 without titration when the molar ratio of the base to the compound of formula I is 0.7 to 1.0 in step (a). In other embodiment, the molar ratio of the base to the compound of formula I is larger than 0.1 and less than 0.7 in step (b), the process just need to add a portion of the base once to maintain pH. The solution is able to be a high-quality and ready-to-use injection suitable for treatment. After lyophilizing the solution, the stable product is able to transport at a temperature below 25° C. As injections, the stable products dissolving in appropriate diluents are able to be used in treatment.

### DETAILED DESCRIPTION OF THE PREFERRED EMBODIMENT

As used herein, the term “hydrate” is used in the conventional sense to include the compounds of formula I and Ia in physical association with water.

As used herein, the term “one mole equivalent” is defined as one mole of carbon dioxide or base source per one mole of active carbapenem (or active drug).

As used herein, the term “active carbapenem” refers to the actual amount of beta-lactam, unstablilized and stabilize carbapenem, and/or alkali-metal salt or alkali earth-metal salt containing carbapenem.

The present invention relates to pharmaceutical compositions which contain the compound of formula I well as salts, stabilized forms and hydrates thereof. Compound of formula I is a carbapenem antibiotic that is particularly useful for intravenous and intramuscular administration.

The process of the present invention generally uses a carbon dioxide source. Preferred sources of carbon dioxide are carbon dioxide (gas, liquid or solid), carbonates and bicarbonates, and more preferably sodium carbonate and sodium bicarbonate, which can be incorporated in the solution, such that an appropriate pH, e.g., about 6.5-8.5, is obtained upon dissolution. The native pH of the monosodium salt of compound I is approximately 5˜6.

Compounds of formula I can be synthesized in accordance with U.S. Pat. No. 5,478,820 issued to Betts, et al. on Dec. 26, 1995, the teachings of which are incorporated herein by reference.

Generally stable product of formula I can be produced by lyophilizing a reconstituted solution blending the compound of formula (I), a carbonate source, and a base. In many instances it is preferred to dissolve the compound of formula I with the carbonate source such as sodium bicarbonate and the base such as sodium hydroxide, in diluents, and then to lyophilize the resulting composition, thus providing a powder composition containing a compound of formula II, or a pharmaceutically acceptable salt, or hydrate thereof.

The carbonate source in gas, solid, liquid or aqueous form of the present invention is selected from the group consisting of carbon dioxide, sodium bicarbonate, potassium bicarbonate, sodium carbonate, potassium carbonate, calcium carbonate, cesium carbonate, magnesium carbonate, lithium carbonate, and a mixture thereof.

The base in gas, solid, liquid or aqueous form of the present invention is selected from the group consisting of sodium hydroxide, lithium hydroxide, potassium hydroxide, calcium hydroxide, barium hydroxide, magnesium hydroxide, lithium methoxide, sodium ethoxide, potassium ethoxide, lithium tert-butoxide, sodium tert-butoxide and potassium tert-butoxide.

The diluent of the present invention is selected from the group consisting of water for injection, sodium chloride injection, bacteriostatic water for injection, and lidocaine HCl injection.

The present invention optimizes a solution process by the following modification: reducing over 10-step manufacturing, simplifying multiple titrations and loosing up the in-process restrictions. The novel process is able to provide better quality of stabilized formulation of carbapenem antibiotics or the same quality (above 94% purity analyzed by HPLC) as quality of other competitors' product (about 94%, purchased from Merk), comprising the steps of:
(a) dissolving a carbonate source and a base in a diluent to form a first solution at a temperature from 0° C. to 25° C., wherein a mole ratio of the carbonate source to the compound of formula I is 0.5 to 1.5, and a mole ratio of the base to the compound of formula I is 0.1 to 1.0; and
(b) mixing the compound of formula I with the first solution at a temperature from −5° C. to 25° C. to form the solution.

Finally, the solution is able to be a high-quality and ready-to-use injection suitable for treatment. After lyophilizing the solution, the stable product is suitable for treatment of bacterial infections after diluting with appropriate diluents.

The process not only simplifies steps of manufacturing and relaxes the restrictions on the manufacturing but also maintains high quality of final products. When the molar ratio of the base to the compound of formula I is 0.7 to 1.0 in step (a), a temperature range of step (a) is from about 0° C. to 25° C., preferably from 0° C. to 15° C. And the pH naturally falls at the appropriate range from about 6.5 to about 8.5 without making actions to maintain pH. The whole process is just simply adding, dissolving, mixing and lyophilizing. In the other embodiment, the molar ratio of the base to the compound of formula I is larger than 0.1 and less than 0.7 in step (a), the simplified process is also able to maintain high quality of final products. And a portion of the base is charged to maintain the pH in the step (b) at a temperature range from about −5° C. to 25° C., preferably from −5° C. to 15° C. The whole process just has to maintain pH with a portion of the base once.

Special preference is given to the compound (Ia) mentioned in the Examples, especially each individual compound.

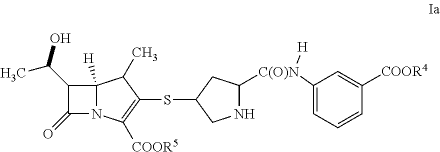

or its pharmaceutically acceptable salt, hydrate or solvate
wherein, R⁴ and R⁵ are independently hydrogen, or C₁-C₆ alkyl, or alkali-metal or alkali earth-metal wherein the alkali-metal or alkali earth-metal is sodium, potassium, lithium, cesium, rubidium, barium, calcium or magnesium.

Yet another objective of the present invention is to provide a simple and commercially viable process for the preparation of compound of the formula IIa, which avoids long-term lyophilization (more than two days)

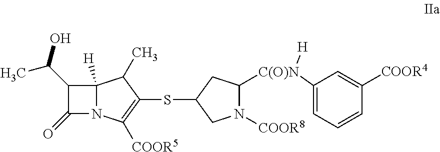

wherein R⁴ and R⁵ are as defined as defined in claim 13, and R⁸ is hydrogen.

The present relates especially to the manufacturing steps mentioned in the following Examples. Although the present invention has been described with reference to the preferred embodiments thereof, it is apparent to those skilled in the art that a variety of modifications and changes may be made without departing from the scope of the present invention which is intended to be defined by the appended claims.

### Example 1

Sodium bicarbonate 1.25 g, 1.0 mole equivalent of sodium bicarbonate/active carbapenem, was added to a reactor already containing 25 ml of water for injection. A sufficient amount of sodium hydroxide, 0.73 mole equivalent of sodium bicarbonate/active carbapenem, was then dissolved thoroughly into the reactor. The solution had a pH range between 10 and 11, and the solution was held at a temperature of from 0° C. to 5° C. Unstable carbapenem 7.56 g of free acid was gradually added to the solution about 30 minutes to ensure complete dissolution, and the solution had a pH at about 8.0.

While maintaining the solution at a temperate between −5° C. and 5° C., the solution was filtered utilizing a filter containing a 0.22 mm filter. The solution was frozen to −40° C. and placed onto the shelves of lyophilizer. Thereafter, the lyophilizer was then operated according to the following cycle:
1. soak at −40° C. shelf temperature for 2 hrs;
2. heat to −20° C. shelf temperature in 40 mins;
3. hold shelf temperature at −20° C. and below 80 mTorr pressure for 24-48 hrs;
4. heat to 10° C. shelf temperature in 5 hrs;
5. heat to 40° C. shelf temperature in 40 mins;
6. hold at 40° C. and below 80 mTorr for 3 hrs;
7. heat to 60° C. shelf temperature in 40 mins;
8. hold at 60° C. and below 80 mTorr for 3 hrs;
9. cool to the shelves to ambient temperature (20° C.-30° C.);

Finally, the final formulation product exhibited a white-powder form and a moisture content of 5%. Table 1 provides the High Performance Liquid Chromatography (HPLC) results in area % of in process samples collected during the formulating of carbapenem antibiotic for this example.

TABLE 1

| HPLC, | | Total | | |
|---|---|---|---|---|
| Area % | carbapenem | degradates | Total dimers | Ring open |
| Bulk drug | 98.94% | 1.06% | 0.27% | 0.24% |
| Prefilter solution | 98.37% | 1.63% | 0.37% | 0.69% |
| Lyophilized product | 95.38% | 4.62% | 1.29% | 2.14% |

### Example 2

The method of Example 2 according to Example 1 to describe the mole ratio of base to the compound of formula I is 0.7 to 1.0 in step (a), and the final solution maintains pH at 6.5 to 8.5.

Sodium bicarbonate 0.98 g, 1.0 mole equivalent of sodium bicarbonate/active carbapenem, was added to a reactor containing 18 ml of water for injection. A sufficient amount of sodium hydroxide, 0.1 mole equivalent of sodium bicarbonate/active carbapenem, was then dissolved thoroughly into the reactor. The solution had a pH at about 9, and then the solution was held at a temperature of from 0° C. to 5° C. Unstable carbapenem 5.8 g of free acid was gradually added to the solution about 30 minutes; at the mean time, a portion of sodium hydroxide were added to achieve 0.73 mole ratio of sodium hydroxide to active carbapenem (appropriate range between 0.7 and 1.0). The final pH was at about 7.7 (appropriate range between about 7.0 and about 8.0).

The solution was filtered utilizing a filter containing a 0.22 mm filter at a temperate of from −5° C. to 5° C. The solution was frozen to −40° C. and placed onto the shelves of Lyophilizer. Thereafter, the lyophilizer was operated according to the same cycle described in Example 1. Table 2 illustrates the High Performance Liquid Chromatography (HPLC) results in area % of in process samples collected during the formulating of carbapenem antibiotic for Example 2.

TABLE 2

| HPLC, | | Total | | |
|---|---|---|---|---|
| Area % | Carbapenem | degradates | Total dimers | Ring open |
| Bulk drug | 98.82% | 1.18% | 0.42% | 0.23% |
| Prefilter soln. | 98.08% | 1.92% | 0.70% | 0.59% |
| Lyophilized product | 96.03% | 3.97% | 1.01% | 1.83% |

### Example 3

The general procedure described in Example 2 was utilized to prepare the formulation of this example. The total mole ratio of sodium hydroxide to active carbapenem achieve to 0.76 (appropriate range from 0.7 to 1.0). The individual amount of reagents is listed in Table 3.

TABLE 3

| | Water | | |
|---|---|---|---|
| Carbapenem | for Injection | NaHCO₃(s) | NaOH |
| 7.94 g | 25 ml | 1.0 mole equivalent | 0.76 mole equivalent |

Table 4 illustrates the High Performance Liquid Chromatography (HPLC) results in area % of in process samples collected during the formulating of carbapenem antibiotic for Example 3.

TABLE 4

| HPLC, | | Total | | |
|---|---|---|---|---|
| Area % | Carbapenem | degradates | Total dimers | Ring open |
| Bulk drug | 98.75% | 1.25% | 0.35% | 0.20% |
| Prefilter soln. | 98.13% | 1.87% | 0.67% | 0.51% |
| Lyophilized product | 97.24% | 2.76% | 1.1% | 0.86% |

### Example 4

At a temperature below 25° C., One mole equivalent of sodium bicarbonate and 0.7 mole equivalent of sodium hydroxide were added into a container. The container was then charged 10 ml of water for injection (WFI), shook thoroughly to form a clear solution within 5 minutes, and mixed with one mole equivalent of ertapenem sodium.

Table 5 illustrates the High Performance Liquid Chromatography (HPLC) results in area % and show the stability of the reconstituted solution produced by the above processes. Purity of the reconstituted solution, above 97%, is 3% higher than commercial products of other competitors. Also, the reconstituted solution exhibits good stability either at a temperature of from about 0° C. to 5° C. or at a temperature of from about 25° C. to 30° C. as shown in Table 5.

TABLE 5

Temp

| | | |
|---|---|---|
| Purity (area %) | 0-5° C. | 25-30° C. |

| | | | |
|---|---|---|---|
| Time | 0 h | 98.24% | 97.42% |
| | 1.5 h | NA | 96.22% |
| | 3 h | 97.42% | 94.51% |
| | 4 h | 97.05% | NA |

A number of embodiments of the invention have been described. Nevertheless, it will be understood that various modifications may be made without departing from the spirit and scope of the invention. Accordingly, other embodiments are within the scope of the following claims.

What is claimed is:

1. A process for manufacturing a solution of a compound of formula I, 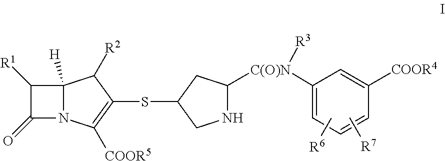
or its pharmaceutically acceptable salt, hydrate or solvate
wherein,
R¹ is 1-hydroxyethyl, 1-fluoroethyl, or hydroxymethyl;
R² and R³ are independently hydrogen, or C₁-C₆ alkyl;
R⁴ and R⁵ are independently hydrogen, C₁-C₆ alkyl, or alkali-metal or alkali earth-metal wherein the alkali-metal or alkali earth-metal is sodium, potassium, lithium, cesium, rubidium, barium, calcium or magnesium;
R⁶ and R⁷ are independently hydrogen, halo, cyano, nitro, hydroxy, carboxy, amino, C₁-C₆ alkylamino, di C₁-C₆ alkylamino, C₁-C₆ alkoxy, C₁-C₆ alkoxycarbonyl, aminosulphonyl, C₁-C₆ alkylaminosulphonyl, di-C₁-C₆ alkylaminosulphonyl, carbamoyl, C₁-C₆ alkylcarbamoyl, trifluoromethyl, sulphonic acid, sulphonic acid, C₁-C₆ alkanoylamino, C₁-C₆ alkanoyl(N—(C₁-C₆)-alkyl)amino, C₁-C₆ alkanesulphonamido, C₁-C₆ alkyl-S(O)ₙ wherein n is 0-2;
comprising the steps of:
(a) dissolving a carbonate source and a base in a diluent to form a first solution at a temperature from 0° C. to 25° C., wherein a mole ratio of the carbonate source to the compound of formula I is 0.5 to 1.5, and a mole ratio of the base to the compound of formula I is 0.1 to 1.0; and
(b) mixing the compound of formula I with the first solution at a temperature from −5° C. to 25° C. to form the solution.

2. The process of claim 1, wherein the carbonate source in gas, solid, liquid or aqueous form is selected from the group consisting of carbon dioxide, sodium bicarbonate, potassium bicarbonate, sodium carbonate, potassium carbonate, calcium carbonate, cesium carbonate, magnesium carbonate, lithium carbonate, and a mixture thereof.

3. The process of claim 1, wherein the base in gas, solid, liquid or aqueous form is selected from the group consisting of sodium hydroxide, lithium hydroxide, potassium hydroxide, calcium hydroxide, barium hydroxide, magnesium hydroxide, lithium methoxide, sodium ethoxide, potassium ethoxide, lithium tert-butoxide, sodium tert-butoxide and potassium tert-butoxide, and a mixture thereof.

4. The process of claim 1, wherein the diluent is selected from the group consisting of water for injection, sodium chloride injection, bacteriostatic water for injection, and lidocaine HCl injection.

5. The process of claim 1, wherein the temperature of the step (a) is preferably from about 0° C. to 15° C. and the temperature of the step (b) is preferably from about −5° C. to 15° C.

6. The process of claim 1, wherein when the mole ratio of the base to the compound of formula I is 0.7 to 1.0 in step (a), the solution of the compound of formula I maintains pH at 6.5 to 8.5.

7. The process of claim 6, further comprising lyophilizing the solution filtered through a micron filter at a temperature from about −5° C. to 15° C. to form a compound of formula II or its pharmaceutically acceptable salt, hydrate or solvate with less than about 10% moisture content 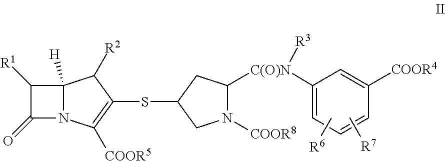
wherein R¹, R², R³, R⁴, R⁵, R⁶ and R⁷ are as defined as defined in claim 1, and R⁸ is hydrogen.

8. The process of claim 6, wherein the solution is able to be a high-quality and ready-to-use injection suitable for treatment.

9. The process of claim 1, wherein when the mole ratio of the base to the compound of formula I is larger than 0.1 and less than 0.7 in step (a), a small portion of the base was added into the solution of the compound of formula I to maintain pH at 6.5 to 8.5.

10. The process of claim 9, further comprising lyophilizing the solution filtered through a micron filter at a temperature from about −5° C. to 15° C. to form a compound of formula II or its pharmaceutically acceptable salt, hydrate or solvate with less than about 10% moisture content 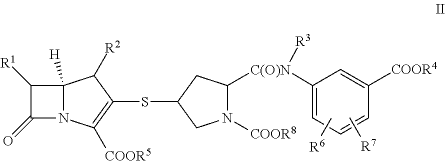
wherein R¹, R², R³, R⁴, R⁵, R⁶ and R⁷ are as defined as defined in claim 1, and R⁸ is hydrogen.

11. A process for manufacturing an solution of a compound of formula Ia, 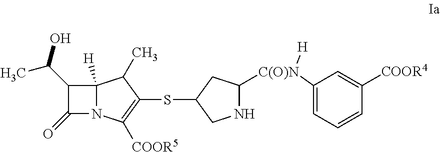
or its pharmaceutically acceptable salt, hydrate or solvate
wherein,
R⁴ and R⁵ are independently hydrogen, or C₁-C₆ alkyl, or alkali-metal or alkali earth-metal wherein the alkali-metal or alkali earth-metal is sodium, potassium, lithium, cesium, rubidium, barium, calcium or magnesium,
comprising the steps of:
(a′) dissolving a carbonate source and a base in a diluent to form a first solution at a temperature from 0° C. to 25° C., wherein a mole ratio of the carbonate source to the compound of formula I is 0.5 to 1.5, and a mole ratio of the base to the compound of formula I is 0.1 to 1.0; and
(b′) mixing the compound of formula Ia with the first solution at a temperature from −5° C. to 25° C. to form the solution.

12. The process of claim 11, wherein the carbonate source is selected from the group consisting of carbon dioxide, sodium bicarbonate, potassium bicarbonate, sodium carbonate, potassium carbonate, calcium carbonate, cesium carbonate, magnesium carbonate, lithium carbonate, and a mixture thereof.

13. The process of claim 11, wherein the base is selected from the group consisting of sodium hydroxide, lithium hydroxide, potassium hydroxide, calcium hydroxide, barium hydroxide, magnesium hydroxide, lithium methoxide, sodium ethoxide, potassium ethoxide, lithium tert-butoxide, sodium tert-butoxide and potassium tert-butoxide and a mixture thereof.

14. The process of claim 11, wherein the diluent is selected from the group consisting of water for injection, sodium chloride injection, bacteriostatic water for injection, and lidocaine HCl injection.

15. The process of claim 11, wherein the temperature of the step (a′) is preferably from about 0° C. to 15° C. and the temperature of the step (b′) is preferably from about −5° C. to 15° C.

16. The process of claim 11, wherein when the mole ratio of the base to the compound of formula I is 0.7 to 1.0 in step (a′), the solution of the compound of formula I maintains pH at 6.5 to 8.5.

17. The process of claim 16, further comprising lyophilizing the solution filtered through a micron filter at a temperature from about −5° C. to 15° C. to form a compound of formula IIa or its pharmaceutically acceptable salt, hydrate or solvate with less than about 10% moisture content 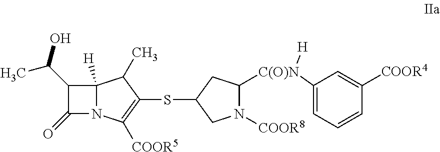
wherein R⁴ and R⁵ are as defined as defined in claim 13, and R⁸ is hydrogen.

18. The process of claim 16, wherein the solution is able to be a high-quality and ready-to-use injection suitable for treatment.

19. The process of claim 11, wherein when the mole ratio of the base to the compound of formula I is larger than 0.1 and less than 0.7 in step (a′), a small portion of the base was added into the solution of the compound of formula I to maintain pH at 6.5 to 8.5.

20. The process of claim 19, further comprising lyophilizing the solution filtered through a micron filter at a temperature from about −5° C. to 15° C. to form a compound of formula IIa or its pharmaceutically acceptable salt, hydrate or solvate with less than about 10% moisture content 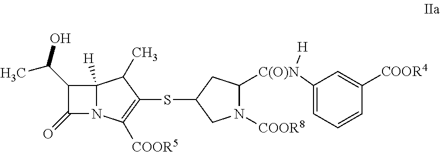
wherein R⁴ and R⁵ are as defined as defined in claim 13, and R⁸ is hydrogen.
